(19) United States
(12) Patent Application Publication
(10) Pub. No.: US 2008/0147031 A1
(43) Pub. Date: Jun. 19, 2008

(54) Array of absorbent articles with indicators

(76) Inventors: Andrew M. Long, Appleton, WI (US); Thomas K. Dixon, Neenah, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/639,976

(22) Filed: Dec. 14, 2006

(51) Int. Cl. A61F 13/42

(52) U.S. Cl. 604/361; 604/358

(57) ABSTRACT

An array of disposable absorbent garments include reagents that can detect the presence of one or more medical conditions through the detection of various substances in urine or feces. The garments of the array may, in addition to or in the alternative, detect physical measurements such as hydration levels or the remaining liquid-capacity of the garment/urine volume in the garment. The garments of the array may vary in size, types of included reagents and/or ability to detect physical measurements.

### BACKGROUND

The stages of life present individuals with certain challenges, one such challenge is dealing with urinary incontinence. As a baby, we cannot help but need a diaper. As an adult, sometimes a situation requires the use of a diaper. During these life stages, persons may be susceptible to diseases and conditions that may adversely affect their overall health if not treated in a timely manner, e.g. diabetes, urinary tract infections, dehydration, and jaundice, just to name a few.

Young children and adults that require assistance from a caregiver with toileting functions or that otherwise need to wear incontinence garments may be susceptible to a host of other conditions that may not be apparent until the condition enters into advanced stages. For example, with respect to jaundice, newborns may be most susceptible. With respect to diabetes or urinary tract infections, adults may be most susceptible. Caregivers of such persons and the wearers themselves typically have very limited or no medical training. Thus, medical conditions such as jaundice, high blood sugar, dehydration, or urinary tract infections often go unnoticed until they have become serious.

It is thus highly desirable to develop a disposable absorbent garment whereby persons can easily detect and/or monitor certain medical conditions. It is further desirable to detect and/or monitor medical conditions that are relatively common for a particular life stage. It is further desirable to detect when an insult has occurred without the need to remove the absorbent garment, and possibly to detect the volume of a urine insult.

### SUMMARY

In one aspect of the invention there is an array of disposable absorbent garments for wearing about the lower torso. The array includes a small garment having a test panel including a reagent. The array also includes a medium garment having a test panel including a reagent. The reagent in the small garment is adapted to react to a first substance, and the reagent in the medium garment is adapted to react to a second substance. The first substance is significantly different than the second substance.

In another aspect of the present invention, there is an array of disposable absorbent garments including a first garment having a first reagent configured to detect, in urine, an abnormal level of a first substance associated with a biliary abnormality after the first reagent contacts said urine. The array further includes a second garment having a second reagent configured to detect, in urine, an abnormal level of a substance associated with a second medical condition after the second reagent contacts said urine. The medical condition may be a urinary tract infection, hematuria, glycosuria, proteinuria, dehydration, or ketonuria.

In yet another aspect of the invention is an array of articles used for detecting a substance in urine including a pad having a liquid permeable bodyside liner, an outer cover, and an absorbent body positioned between the bodyside liner and the outer cover, the pad adapted to collect urine. The pad further includes a test panel having a chemical reagent capable of detecting the substance. The array also has a garment adapted to support the pad against the body, the garment including a window through which the pad test panel may be observed.

### BRIEF DESCRIPTION OF DRAWINGS

Refer now to the figures, which are exemplary, not limiting, and wherein like elements are numbered alike.

FIG. 1 representatively shows a top plan view of one embodiment of an absorbent article of the present invention shown in a stretched and laid flat condition, with the surface of the article that contacts the skin of the wearer facing the viewer;

FIG. 2 is a side perspective view of an embodiment of another article of the present invention shown in the form of a pair of pants having a mechanical fastening system shown fastened on one side of the pants and unfastened on the other side of the pants;

FIG. 3 is a schematic view of one embodiment of a visual indicator of the present invention, for use on the absorbent article of FIGS. 1 and 2;

FIG. 4 is one embodiment of an array of the present invention; and

FIG. 5 is another embodiment of an array of the present invention.

### DETAILED DESCRIPTION

Disclosed herein are absorbent articles and systems for determining the existence of an insult, for estimating a volume of a liquid in absorbent articles, and/or for identifying one or more substances in urine.

### Definitions:

As used herein, the term “array” is meant to include a collection of disposable absorbent articles manufactured by or for a single business entity (including all divisions, subsidiaries, parent companies, and assigns) and/or its licensees. The array of disposable articles may be sold under different trademarks or brands, unless otherwise specified in the claims.

As used herein, the term “life stage” is meant to generally refer to the stages of human life. Persons in particular life stages include newborns, infants, toddlers, children, and adults. For the average population, such life stages may be determined by body weight or by age. (This definition does not take into account the mental state or capacity of a person.) Newborns may weigh anywhere from about 0.45 kilograms to about 5.44 kilograms. Infants may weigh anywhere from about 2.27 kilograms to about 12 kilograms. Toddlers may weigh anywhere from about 10 kilograms to about 35 kilograms. Young adults and adults may weigh anywhere from about 36 kilograms upward. Of course, there is much overlap in weights between the life stages, and such ranges are not meant to limit the present invention. With respect to age, newborns may be from 0 to about 3 months (depending on whether they were prematurely born). Infants are from about 2 months to about 12 months. Toddlers may be about 12 months to about 3 years. Children may be about 3 years to about 14 years. Adults may be about 14 years upward. At each weight range and/or age, persons may be more susceptible to certain medical conditions such as diabetes, urinary tract infections, etc. However, if a person is not more susceptible to such conditions, it may be that at certain weight ranges and/or ages, some medical conditions are of more interest or concern.

The term “substance” is used herein to refer to any substance in urine, the presence or absence of which, that has a known relationship to an individual's health. Hence, the substance may be selected to indicate the presence or absence of a pathological disorder or to indicate or monitor an individual's physiological condition. In the latter case, it is the concentration of the substance which generally is of interest. The term “pathological disorder” is used herein to mean a disease or reaction caused by a foreign substance which elicits an immune response or disrupts a normal body function. The term “physiological condition” refers to the state of organ functions and their interrelationships which impact overall health. Accordingly, the phrase “presence of a substance in urine” is meant to encompass the presence or absence of the substance. If the substance is present, the visual indication additionally can provide information on or an estimate of the concentration of the substance. All of these variations are deemed to come within the scope of the present invention.

The term “visual indication” is used herein to mean any indication capable of being read, i.e., capable of being observed visually and understood or interpreted without the need for a special instrument or apparatus. As used herein, the term “special instrument or apparatus” means a device designed or constructed for the specific purpose of reading and/or interpreting the visual indication or an existing device inherently capable of so doing. The term does not include a device used to correct defects of vision, such as glasses and contact lenses. The term also does not include a calibration scale or color chart to relate color to substance concentration, or other instructional, informational, or interpretational aids. While a color change or the appearance or disappearance of a color are the most common forms of a visual indication, the term is not intended to be limited thereto. For example, the visual indication could be a symbol, alphanumeric character, or the like.

As used herein, “glycosuria” is defined as the presence of glucose in urine. “Biliary abnormality” is defined as any inflammatory, fibrotic, or obstructive disorder of the liver, bile ducts, or pancreas that produces elevation in serum total, direct, or indirect bilirubin levels; including, for example, cholelithiasis, cholecystitis, Gilbert's disease, hepatic cancer, cholangiocarcinoma, pancreatic cancer, and hepatitis. When serum bilirubin concentrations are elevated, bilirubin can appear in the urine, as can urobilinogen. “Ketonuria” is defined as the presence of ketones (ketone bodies), for example beta hydroxybutyrate, in urine. “Proteinuria” is defined as the presence of protein, for example albumin, in urine. “Hematuria” is defined as the presence of red blood cells present in urine, not simply those amounts considered clinically significant.

The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced items. Unless defined otherwise, technical and scientific terms used herein have the same meaning as is commonly understood by one of skill in the art to which this invention belongs. The modifier “about” used in connection with a quantity is inclusive of the stated value and has the meaning dictated by the context, (e.g., includes the degree of error associated with measurement of the particular quantity).

### Exemplary Absorbent Products:

The disposable absorbent garments in an array of the present invention are suitably diapers and/or pants. One particular embodiment of a diaper and a pant is described herein, though it is contemplated that many variations of the diapers and pants may be used in an array of the present invention.

### A. Diaper

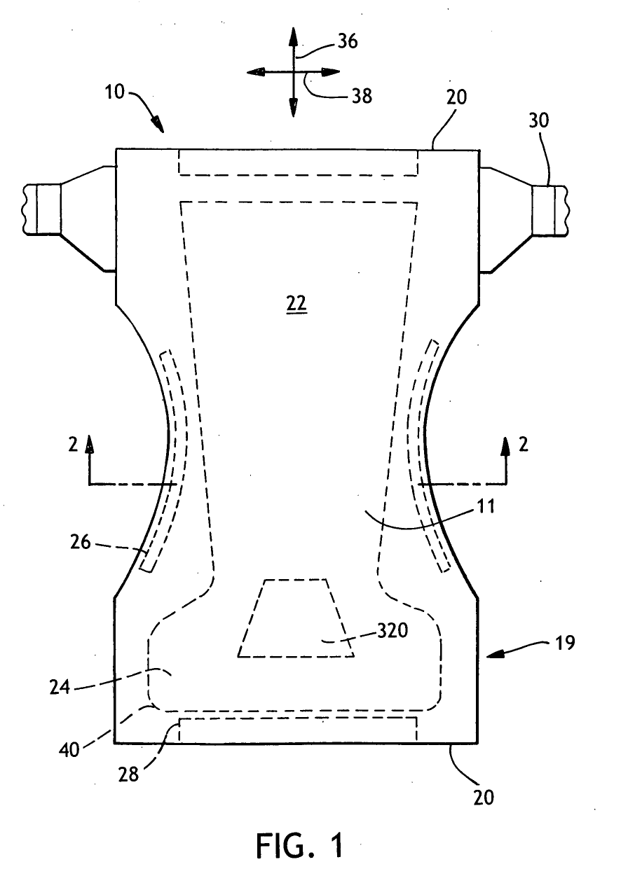

Referring now to FIG. 1, in one embodiment, the absorbent article is a disposable diaper 10 comprising a backsheet or outer cover 20, a liquid permeable topsheet or bodyside liner 22 positioned in facing relation with the outer cover 20, and an absorbent body 24, such as an absorbent pad, which is located between the bodyside liner 22 and the outer cover 20. The outer cover 20 defines a length and a width that, in the illustrated aspect, coincide with the length and width of the diaper 10. The absorbent body 24 may define a length and width that are less than the length and width of the outer cover 20, respectively. Thus, marginal portions of the diaper 10, such as marginal sections of the outer cover 20, may extend past the terminal edges of the absorbent body 24. In the illustrated aspects, for example, the outer cover 20 extends outwardly beyond the terminal marginal edges of the absorbent body 24 to form side margins and end margins of the diaper 10. The bodyside liner 22 is generally coextensive with the outer cover 20 but may optionally cover an area that is larger or smaller than the area of the outer cover 20, as desired. In other words, the bodyside liner 22 is connected in superposed relation to the outer cover 20. The outer cover 20 and bodyside liner 22 are intended to face the garment and body of the wearer, respectively, while in use. A front panel 19 may have a test panel 320 on the outer cover 20, as described herein.

To provide improved fit and to help reduce leakage of body exudates from the diaper 10, the diaper side margins and end margins can be elasticized with suitable elastic members, such as single or multiple strands of elastic. As representatively illustrated in FIG. 1, the diaper 10 may include leg elastics 26 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics 28 are configured to provide a resilient comfortably close fit around the waist of the wearer. In FIG. 1, the elastic members are illustrated in their uncontracted, stretched condition for the purpose of clarity.

Fastening means, such as hook and loop fasteners 30, can be employed to secure the diaper 10 on a wearer. Alternatively, other fastening means, such as buttons, pins, snaps, adhesive tape fasteners, cohesives, mushroom-and-loop fasteners, a belt, and so forth, as well as combinations comprising at least one of the foregoing fasteners may be employed. Additionally, more than two fasteners can be provided, particularly if the diaper 10 is to be provided in a prefastened configuration.

The diaper 10 may further include other layers between the absorbent body 24 and the bodyside liner 22 or outer cover 20.

The diaper 10 may be of various suitable shapes. For example, the diaper may have an overall rectangular shape, T-shape or an approximately hourglass shape. In the shown aspect, the diaper 10 has a generally I-shape. The diaper 10 further defines a longitudinal direction 36 and a lateral direction 38. Other suitable diaper components that may be incorporated on absorbent articles include containment flaps, waist flaps, elastomeric side panels, and the like. Examples of possible diaper configurations are described in U.S. Pat. No. 4,798,603 issued Jan. 17, 1989, to Meyer et al.; U.S. Pat. No. 5,176,668 issued Jan. 5, 1993, to Bemardin; U.S. Pat. No. 5,192,606 issued Mar. 9, 1993, to Proxmire et al., and U.S. Pat. No. 5,509,915 issued Apr. 23, 1996 to Hanson et al.

The outer cover 20 of the diaper 10 can comprise any material used for such applications, such as a substantially vapor permeable material. The permeability of the outer cover 20 can be configured to enhance the breathability of the diaper 10 and to reduce the hydration of the wearer's skin during use without allowing excessive condensation of vapor, such as urine, on the garment facing surface of the outer cover 20 that can undesirably dampen the wearer's clothes.

As stated above, the outer cover 20 can comprise any material used for such applications, and desirably comprises materials that either directly provide the above desired levels of liquid impermeability and air permeability and/or materials that can be modified or treated in some manner to provide such levels. The outer cover 20 can be a nonwoven fibrous web constructed to provide the required level of liquid impermeability. For example, a nonwoven web comprising spunbond and/or meltblown polymer fibers may be selectively treated with a water repellent coating and/or laminated with a liquid impermeable, vapor permeable polymer film to provide the outer cover 20. In another embodiment, the outer cover 20 can include a nonwoven web comprising a plurality of randomly deposited hydrophobic thermoplastic meltblown fibers that are sufficiently bonded or otherwise connected to one another to provide a substantially vapor permeable and substantially liquid impermeable web. The outer cover 20 may also include a vapor permeable nonwoven layer that has been partially coated or otherwise configured to provide liquid impermeability in selected areas. In yet another example, the outer cover 20 is provided by an extensible material. Further, the outer cover 20 material can have stretch in the longitudinal 36 and/or lateral 38 directions. When the outer cover 20 is made from extensible or stretchable materials, the diaper 10 provides additional benefits to the wearer including improved fit.

The bodyside liner 22, employed to help isolate the wearer's skin from liquids held in the absorbent body 24, can define a compliant, soft feeling, nonirritating to the wearer's skin bodyfacing surface. Further, the bodyside liner 22 can be less hydrophilic than the absorbent body 24, to present a relatively dry surface to the wearer, and can be sufficiently porous to be liquid permeable, permitting liquid to readily penetrate through its thickness. A suitable bodyside liner 22 can be manufactured from a wide selection of web materials, such as porous foams, reticulated foams, apertured plastic films, natural fibers (for example, wood or cotton fibers), synthetic fibers (for example, polyester or polypropylene fibers), and the like, as well as a combination of materials comprising at least one of the foregoing materials.

Various woven and nonwoven fabrics can be used for the bodyside liner 22. For example, the bodyside liner 22 can comprise a meltblown or spunbond web (e.g., of polyolefin fibers), a bonded-carded web (e.g., of natural and/or synthetic fibers), a substantially hydrophobic material (e.g., treated with a surfactant or otherwise processed to impart a desired level of wetability and hydrophilicity), and the like, as well as combinations comprising at least one of the foregoing.

The liquid-absorbent layer or absorbent body 24 of the diaper 10 can comprise a matrix of hydrophilic fibers, such as a fibrous web of cellulosic fibers, mixed with particles of a high absorbency material (such as the material commonly known as superabsorbent material). The wood pulp fluff may be exchanged with synthetic, polymeric, meltblown fibers, and the like, as well as a combination comprising at least one of the foregoing. The superabsorbent particles can be substantially homogeneously mixed with the hydrophilic fibers or may be nonuniformly mixed. Alternatively, the absorbent body 24 can include a laminate of fibrous webs and superabsorbent material and/or a suitable matrix for maintaining the superabsorbent material in a localized area.

The high absorbency material (e.g., superabsorbent) can be natural, synthetic, and modified natural polymers and materials; inorganic materials (such as silica gels); organic compounds (such as crosslinked polymers); and the like, as well as combinations comprising at least one of the foregoing. The term “crosslinked” refers to methods for effectively rendering normally water-soluble materials substantially water insoluble but swellable. Such methods include, but are not limited to, physical entanglement, crystalline domains, covalent bonds, ionic complexes and associations, hydrophilic associations such as hydrogen bonding, and/or hydrophobic associations or Van der Waals forces. Examples of high absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and the like, as well as copolymers and combinations comprising at least one of the foregoing. Further polymers suitable for use in the absorbent body 24 include, but are not limited to, polymers (natural and modified natural), such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum, and so forth. Mixtures of natural and wholly or partially synthetic absorbent polymers can also be useful. Similarly useful are various copolymers and, combinations comprising at least one of any of the above high-absorbency materials.

Optionally, the absorbent body 24 may further comprise a support (e.g., a substantially hydrophilic tissue or nonwoven wrapsheet (not illustrated)) to help maintain the integrity of the structure of the absorbent body 24. The tissue wrapsheet can be placed about the web/sheet of high-absorbency material and/or fibers, optionally over at least one or both major facing surfaces thereof. The tissue wrapsheet can comprise an absorbent cellulosic material, such as creped wadding or a high wet-strength tissue. The tissue wrapsheet can optionally be configured to provide a wicking layer that helps to rapidly distribute liquid over the mass of absorbent fibers constituting the absorbent body 24.

To improve the overall liquid uptake and air exchange, the diaper 10 may further include a porous, liquid-permeable layer of surge management material (not shown). The surge management layer is typically less hydrophilic than the absorbent body 24, and can have an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the diaper 10 positioned against the wearer's skin, thereby reducing the feeling of wetness by the wearer. The structure of the surge management layer can also enhances the air exchange within the diaper 10.

### B. Pant

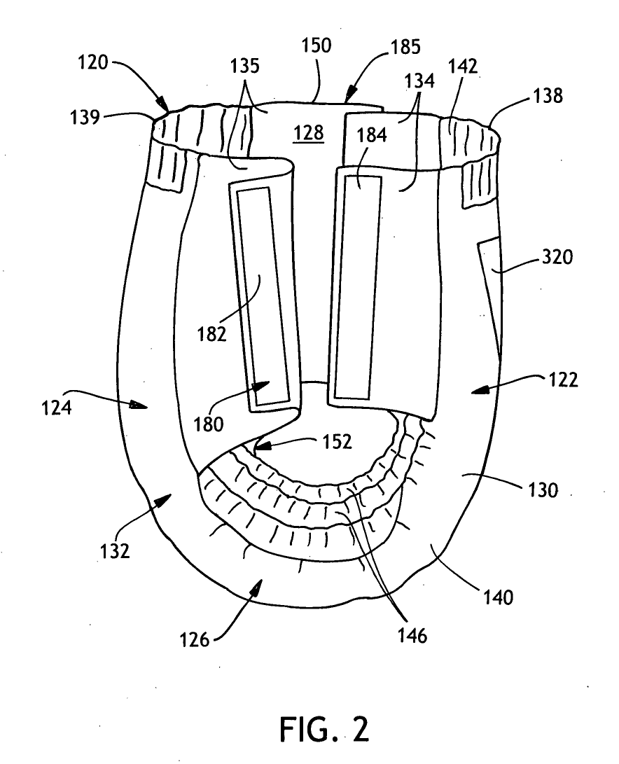

Referring now to the drawings and in particular to FIG. 2, an article of the present invention is shown therein in the form of pants and is indicated in its entirety by the reference numeral 120.

By way of illustration only, various materials and methods for constructing the pants 20 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al.; U.S. Pat. No. 7,018,369 issued Mar. 28, 2006 to Van Gompel et al.; U.S. Pat. No. 6,497,695 issued Dec. 24, 2002 to Bruemmer-Prestley, et al.; U.S. Pat. No. 6,569,139 issued May 27, 2003 to Datta et al.; and U.S. Pat. No. 7,077,834 issued Jul. 18, 2006 to Bishop et al. which are incorporated herein by reference to the extent that they are consistent with the present invention.

The pair of pants 120 is illustrated in FIG. 2 in a partially fastened condition and comprises a front waist region 122, a back waist region 124, a crotch region 126 interconnecting the front and back waist regions, an inner surface 128 configured for contiguous relationship with the wearer, and an outer surface 30 opposite the inner surface. The front waist region 122 is contiguous with the front waist edge 138, and the back waist region 124 is contiguous with the back waist edge 139. A front waist region 122 may have a test panel 320 as described herein.

The illustrated pants 120 comprises a central absorbent assembly, generally indicated at 132, which when laid flat can be rectangular or any other desired shape, a pair of laterally opposite front side panels 134 extending outward therefrom at the front waist region 122 and a pair of laterally opposite back side panels 135 extending outward therefrom at the back waist region 124. The absorbent assembly 132 and side panels 134, 135 may comprise two or more separate elements, as shown in FIG. 2, or they may be integrally formed. The central absorbent assembly 132 of the illustrated embodiment comprises an outer cover 140 and a bodyside liner 142 connected to the outer cover in a superposed relation by suitable means such as adhesives, ultrasonic bonds, thermal bonds or other conventional techniques. An absorbent structure is disposed between the outer cover and the bodyside liner. Desirably, a pair of containment flaps 146 is secured to the bodyside liner 42 for inhibiting the lateral flow of body exudates.

With the pants 120 in the fastened position as partially illustrated in FIG. 1, the front and back side panels 134, 135 are connected together by a fastening system 180 to define a three-dimensional pants configuration having a waist opening 150 and a pair of leg openings 152. The front waist region 122 comprises the portion of the pants 120 which, when worn, is positioned on the front of the wearer while the back waist region 124 comprises the portion of the pants which is positioned on the back of the wearer. The crotch region 126 of the pants 120 comprises the portion of the pants 20 which is positioned between the legs of the wearer and covers the lower torso of the wearer. The front and back side panels 134 and 135 comprise the portions of the pants 20 which, when worn, are positioned on the hips of the wearer. The waist edges 138 and 139 of the pants 20 are configured to encircle the waist of the wearer and together define the waist opening 150. Portions of the side edges 136 in the crotch region 126 generally define the leg openings 152.

The central absorbent assembly 132 is configured to contain and/or absorb exudates discharged from the wearer. A flap elastic member can be operatively joined with each containment flap 146 in any suitable manner as is well known in the art. The elasticized containment flaps 146 define a partially unattached edge which assumes an upright configuration in at least the crotch region 126 of the pants 120 to form a seal against the wearer's body. Suitable constructions and arrangements for the containment flaps 46 are generally well known to those skilled in the art and are described in U.S. Pat. No. 4,704,116 issued Nov. 3, 1987 to Enloe, which is incorporated herein by reference.

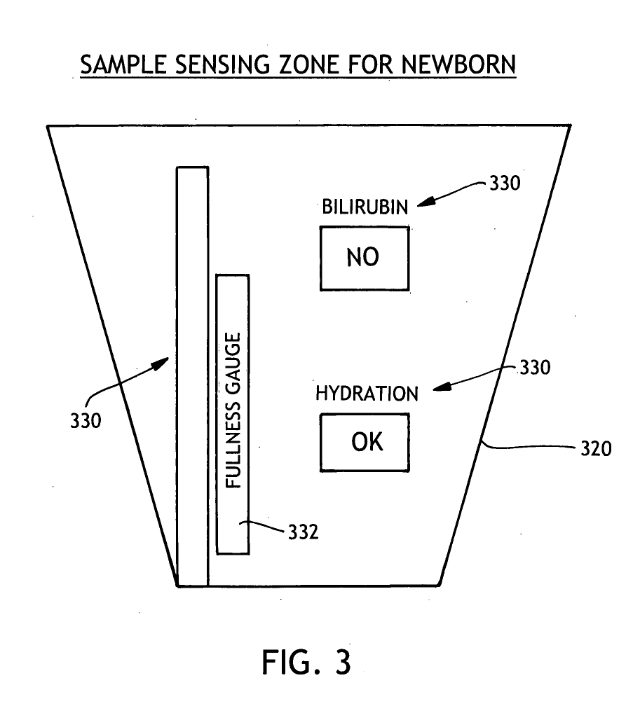

To further enhance containment and/or absorption of body exudates, the pants 20 also suitably includes a front waist elastic member 154 (FIG. 3), a rear waist elastic member 156, and leg elastic members 158, as are known to those skilled in the art.

The flap elastic members 153, the waist elastic members 154 and 156, and the leg elastic members 58 can be formed of any suitable elastic material. As is well known to those skilled in the art, suitable elastic materials include sheets, strands or ribbons of natural rubber, synthetic rubber, or thermoplastic elastomeric polymers.

The outer cover 140 suitably comprises a material which may be substantially liquid impermeable. The outer cover 140 can be a single layer of liquid impermeable material, but more suitably comprises a multi-layered laminate structure in which at least one of the layers is liquid impermeable. For instance, the outer cover 140 can include a liquid permeable outer layer and a liquid impermeable inner layer that are suitably joined together by a laminate adhesive, ultrasonic bonds, thermal bonds, or the like. The outer layer may also be made of those materials of which the liquid permeable bodyside liner 142 is made. While it is not a necessity for the outer layer to be liquid permeable, it is suitable that it provides a relatively cloth-like texture to the wearer.

The inner layer of the outer cover 140 can be both liquid and vapor impermeable, or it may be liquid impermeable and vapor permeable. The inner layer can be manufactured from a thin plastic film, although other flexible liquid impermeable materials may also be used. The inner layer, or the liquid impermeable outer cover 140 when a single layer, prevents waste material from wetting articles, such as bed sheets and clothing, as well as the wearer and/or caregiver.

If the outer cover 140 is a single layer of material, it can be embossed and/or matte finished to provide a more cloth-like appearance. As earlier mentioned, the liquid impermeable material can permit vapors to escape from the interior of the disposable absorbent article, while still preventing liquids from passing through the outer cover 40. A suitable “breathable” material is composed of a microporous polymer film or a nonwoven fabric that has been coated or otherwise treated to impart a desired level of liquid impermeability.

The liquid permeable bodyside liner 142 is illustrated as overlying the outer cover 140, and absorbent core 144, and may, but need not have the same dimensions as the outer cover 140. Further, the bodyside liner 142 can be less hydrophilic than the absorbent structure 144 to present a relatively dry surface to the wearer and permit liquid to readily penetrate through its thickness. Alternatively, the bodyside liner 142 can be more hydrophilic or can have essentially the same affinity for moisture as the absorbent structure 144 to present a relatively wet surface to the wearer to increase the sensation of being wet. This wet sensation can be useful as a tactile means for alerting the wearer that an insult has occurred. The hydrophilic/hydrophobic properties can be varied across the length, width and/or depth of the bodyside liner 142 and absorbent structure 144 to achieve the desired wetness sensation or leakage performance. The bodyside liner 142 can be manufactured from a wide selection of web materials, such as those described for diaper 20 herein.

As noted previously, the illustrated pants 120 have front and back side panels 134 and 135 disposed on each side of the absorbent assembly 132. The side panels 134, 135 can be permanently bonded along seams to the central absorbent assembly 132 in the respective front and back waist regions 122 and 124. The side panels 134 and 135 may be bonded to the absorbent assembly 32 using attachment means known to those skilled in the art such as adhesive, thermal or ultrasonic bonding. Alternatively, the side panels 134 and 135 can be formed as an integral portion of a component of the absorbent assembly 32.

The side panels 134, 135 suitably, although not necessarily, comprise an elastic material capable of stretching in a direction generally parallel to the transverse axis of the pants 120. Suitable elastic materials, as well as one process of incorporating elastic side panels into pants, are described in the following U.S. Pat. No. 4,940,464 issued Jul. 10, 1990 to Van Gompel et al.; U.S. Pat. No. 5,224,405 issued Jul. 6, 1993 to Pohjola; U.S. Pat. No. 5,104,116 issued Apr. 14, 1992 to Pohjola; and U.S. Pat. No. 5,046,272 issued Sep. 10, 1991 to Vogt et al.; all of which are incorporated herein by reference. Alternatively, the side panel material may comprise other woven or nonwoven materials, such as those described above as being suitable for the outer cover 140 or bodyside liner 142; mechanically pre-strained composites; or stretchable but inelastic materials.

The fastening system 180 comprises laterally opposite first fastening components 182 adapted for refastenable engagement to corresponding second fastening components 184. In one embodiment, a front or outer surface of each of the fastening components 182, 184 comprises a plurality of engaging elements. The engaging elements of the first fastening components 182 may be adapted to repeatedly engage and disengage corresponding engaging elements of the second fastening components 184 to releasably secure the pants 120 in its three-dimensional configuration.

The fastening components 182, 184 can comprise any refastenable fasteners suitable for absorbent articles, such as adhesive fasteners, cohesive fasteners, mechanical fasteners, or the like, as described herein for the diaper 10.

The liquid-absorbent layer or absorbent structure (not shown) can be any structure which is generally compressible, conformable, non-irritating to the wearer's skin and capable of absorbing and retaining liquid body exudates, and may be manufactured in a wide variety of sizes and shapes, and from a wide variety of absorbent materials commonly used in the art. The liquid-absorbent layer may be the same or similar to that described for absorbent body 24 of the diaper 10.

### Chemistry:

Reagents that react with a substance which may be found in urine may be included in an absorbent product. For instance, various chemicals may be used to detect a substance obtained from a urine insult in a disposable and absorbent diaper, pant, or pad.

In chemical terms, the substance present in the urine reacts with, acts on, or is acted upon by (or is not present to react with, act on, or be acted upon by) a chemical reagent to provide visual indication of the presence of the substance. Optionally, the concentration of the substance will also be shown. If present, the substance is referred to herein for the sake of simplicity as interacting with or activating the chemical reagent. For convenience, the discharge of an amount of a substance sufficient to interact with the chemical reagent is referred to herein as an “insult.” Nonlimiting examples of substances include: hydrogen ions; ion aggregate (i.e., total ion concentration); nitrite; leukocytes; glucose; ketones; blood; phenylalanine; bilirubin; urobilinogen; protein; albumin; specific enzymes, such as lactate dehydrogenase; and urine-excreted drugs. In certain embodiments of the present invention, the substance will be one of the following: hydrogen ion, ion aggregate (i.e., total ion concentration), nitrite, leukocytes, glucose, ketones, blood, phenylalanine, bilirubin, and urobilinogen.

The visual indication may be independent of the total quantity of the substance to which the chemical reagent is exposed, provided only that a sufficient quantity is present in the urine. In general, the minimum amount is simply that amount which is necessary to activate the chemical reagent. However, the minimum amount is in part dependent upon the concentration in the urine of the specific substance of interest.

As with many analytical procedures, there generally will be a minimum or threshold level for many of the chemical reagents which come within the scope of the present invention. That is, the amount or concentration of the substance of interest can only be detected by the chemical reagent when such amount or concentration is above the threshold level of detection. Thus, the threshold level is the minimum amount of substance which is necessary for a given chemical reagent and will be known or readily determined by those having ordinary skill in the art without undue experimentation.

The maximum amount of substance, on the other hand, is related to the maximum amount of urine which the disposable absorbent product was designed to absorb. Many substances of interest are excreted in relatively small quantities in comparison with the total amount of urine. Once the substance has interacted or reacted with the chemical reagent, discharges of additional substance-containing urine generally will have little or no effect on the visual indication provided by the chemical reagent. Thus, the maximum amount of substance is that amount of substance contained in the total amount of bodily excrement present in the disposable absorbent product. The maximum amount of bodily excrement, in turn, is the maximum amount which the disposable absorbent product was designed to absorb.

Desirably, the chemical reagents employed in the present invention are end point-based. As used herein, the term “end point” means the point marking the completion of the process which provides the required visual indication. Consequently, the visual indication has not been provided until the end point of the chemical reagents has been reached. Thus, instead of having to be read at a specific time after the chemical reagents have interacted with the substance, the visual indication can be read over an extended period of time after the end point has been reached. However, it may be necessary to use time-dependents chemical reagents for certain tests, if an end-point based method is not available.

The chemical reagent has three characteristics. First, the chemical reagent is adapted to provide the visual indication as a result of interacting with a substance. This means that there is no need to submit the disposable absorbent product or the chemical reagent to further processing steps of any kind. For example, the disposable absorbent product does not need to be taken to a health care professional for interpretation, or submitted to a laboratory for further testing or analysis. However, the visual indication may or may not be capable of being read without a special instrument or apparatus, such as a color code. For instance, one may need to compare the resulting color from an interaction between the urine and the chemical reagent in order to determine the test result. Desirably, for convenience sake, such a color code would be printed at the surface of the disposable absorbent garment on a test panel 320, as described herein.

Desirably, the end point of the chemical reagent is adapted to be reached within a period of time which begins with the first insult and ends when the disposable absorbent product is discarded. Most suitably, the end point will be reached shortly after the first insult, e.g., within about 30 seconds.

The chemical reagent may be adapted to have a stable end-point, such that the visual indication remains valid within a period of time which begins when the end point is reached and ends no sooner than when the disposable absorbent product is discarded. Ideally, the end point will be stable up to the time the disposable absorbent product is discarded. Optionally, one may need to read the test result within a certain period of time after the insult occurs, but prior to when the disposable absorbent garment is discarded. In this case, it is suitable that the end point not change after a period of hours, for example about 8 hours. This would allow a person to wake after sleeping and still be able to read the end point result.

Desirably, each disposable absorbent product of the present invention, notwithstanding the presence of a chemical reagent, still functions as an absorbent product. Thus, the product is intended to absorb urine in the same manner as a product which lacks the chemical reagent but which otherwise is functionally similar to the disposable absorbent product of the present invention. For example, if a disposable absorbent product of the present invention were a diaper, it still would function as a diaper. Because the disposable absorbent product also includes the chemical reagent, however, the product serves two functions.

The disposable absorbent product of the present invention is adapted to be handled after manufacture in the same manner as a product which lacks the chemical reagent but which otherwise is functionally similar to the disposable absorbent product. That is, the presence of the chemical reagent in the disposable absorbent product desirably does not require special packaging materials, such as light-blocking films; special packaging conditions, such as packaging under an atmosphere of nitrogen or the like; special storage and transportation, or distribution, conditions, such as refrigeration; or the like. Thus, a disposable absorbent product of the present invention ideally will be packaged, stored, shipped, and distributed, i.e., handled after manufacture, in much the same manner as related goods not containing the chemical reagent. However, such factors as shelf life limitations, the avoidance of high storage temperatures, and compliance with government regulations may be unavoidable.

In general, the chemical reagent may be located anywhere within the wearable disposable absorbent product where contact with urine can occur. Desirably, the chemical reagent will be located primarily in the portion of the product which is most likely to be exposed to urine, i.e., an area of the product which commonly is referred to as the target zone.

The chemical reagent may be provided in the liquid-absorbent layer (e.g. absorbent body 24) of a disposable absorbent article by any of a number of methods commonly known in the art. Examples thereof include the application of powder in appropriate areas of the liquid-absorbent layer, the weaving of fibers impregnated with the appropriate reagents in the appropriate areas of the liquid-absorbent layer, and the like. These reagents can be provided during the manufacturing process of the diaper in the liquid-absorbent layer in the crotch area between the liner (the layer next to the skin) of the diaper and the liquid-absorbent layer in such a manner that color changes associated with the various reactions are easily visible through the liner or body-facing surface on removal of the diaper, or through the garment-facing surface or outer cover even before removal of the disposable absorbent garment. A change in the color of any of the reagents indicates the presence of an abnormal substance or substance levels in the insult.

Rather than impregnate the liquid absorbent layer, it is further contemplated that the chemical reagent may be included in a lateral flow device. In general, lateral flow devices (not shown) are made from a carrier sheet having three segments: an edge margin, a constituent zone, and a diagnostic area. A plurality of constituents is disposed in the defined zone between the edge margin and the diagnostic area. The diagnostic area is isolated from moisture so that when any liquid is absorbed at the edge margin of the carrier sheet, the liquid travels through the constituent zone and into the diagnostic area. As the liquid is absorbed by the diagnostic area, the constituents are carried into the diagnostic area at different speeds. For example, if the plurality of constituents defines a line at a surface of the defined zone, liquid moving from the edge margin to the diagnostic area will transport the constituents. The constituents, being particles of varying size, will travel at different speeds for different distances, depending in part on whether certain substances are present in the liquid. Thus, the diagnostic area will show when a substance is present or not present depending on where the constituents remain after they are moved as far as possible into the diagnostic area. See, Absorbent Articles Including A Body Fluid Signaling Device, Kimberly-Clark Docket 64103156US01, Filed Dec. 14, 2006, to Nhan et al., incorporated by reference to the extent it is consistent with the present invention.

Regardless of whether a lateral flow device is present in the absorbent product, most suitably the result of the interaction between an insult and the chemical reagent is viewable from the garment facing surface of the disposable absorbent article, such as diaper 10 (FIG. 1) and pant 120 (FIG. 2). Desirably, the result of any interaction between a liquid or substance and the chemical reagent is viewable at a test panel 320 such as that shown by way of example in FIG. 3. The test panel 320 may contain test results for one or more substances, and may be of any shape. Desirably, the test panel 320 has a background color or colors that is either neutral to the test result(s), or assists the caregiver or wearer of the disposable absorbent article in reading the test result(s).

At least one chemical reagent may be provided in the liquid-absorbent layer or in a lateral flow device; examples thereof include glucose oxidase, at a concentration of about 16.3% by weight (this may be obtained from, for example, the fungus Aspergillus niger); peroxidase, at a concentration of about 0.6% by weight; potassium iodide, at a concentration of about 7.0% by weight; cumene-hydroperoxide tetramethylbenzidine (this is preferably cumene-hydroperoxide 3,3′,5,5′, tetramethylbenzidine, although other methyl group substitution configurations may be possible), at a concentration of about 22.5% by weight; naphthyl ester, at a concentration of about 0.4% by weight; diazonium salt, at a concentration of about 0.2% by weight; arsenilic acid tetrahydrobenzo (h) quinolin (this is preferably p-arsenilic acid 1, 2, 3, 4 tetrahydrobenzo (h) quinolin, although other substitution configurations may be possible), at a concentration of about 1.4% by weight; methyl red, at a concentration of about 0.2% by weight; bromthymol blue, at a concentration of about 2.8% by weight; tetrabromphenol blue, at a concentration of about 0.3% by weight; sodium nitroprusside, at a concentration of about 7.1% by weight; dichloroaniline diazonium salt (this is preferably 2,4-dichloroaniline diazonium salt, although other substitution configurations may be possible), at a concentration of about 0.4% by weight; diethylaminobenzaldehyde (this is preferably p-diethylaminobenzaldehyde, although other substitution configurations may be possible), at a concentration of about 2.9% by weight; and polyacid, at a concentration of about 1.2% by weight. It should be understood that other concentrations of these or similar reagents may also be employed. These reagents, the substances with which they react, and the color changes associated with these reactions are listed in Table 1. However, the reagents of Table 1 are exemplary only, and are not intended to limit the scope of the invention.

TABLE 1

| | | Abnormal | | |
|---|---|---|---|---|
| | | Color | | |
| | | Changes | | |
| Substance | Chemical Reagent | Low | Medium | High |
| Glucose | 16.3% Glucose Oxidase; | Khaki | Brown | Dark |
| | 0.6% Peroxidase (peroxidase); | | | Brown |
| | 7.0% Potassium iodide | | | |
| Blood | 22.5% Cumene- | Lt. Green | Med. Green | Dark |
| | hydroperoxide; 3,3′ 5,5′ | | | Blue |
| | tetramethyl-benzidine | | | |
| Leukocytes | .04% Naphthyl ester; | Beige/ | Lavender | Purple |
| | 0.2% Diazonium salt | Pink | | |
| Nitrite | 1.4% p-Arsanilic (arsenilic) | Lt. Pink | Med. Pink | Pink |
| | acid; 1,2,3,4 Tetrahydrobenzo | | | |
| | (h) quinolin | | | |
| pH | 0.2% Methyl red; | Orange/ | Lt. Green | Green/ |
| | 2.8% Bromthymol blue | Yellow | | Blue |
| Protein | 0.3% Tetrabromphenol blue | Yellow/ | Green | Green- |
| | | Green | | Blue |
| Ketone | 7.1% Sodium nitroprusside | Pink | Purple | Deep |
| | | | | Purple |
| Bilirubin | 0.4% 2,4-dichloroaniline | Cream | Beige Pink | Lavender |
| | diazonium salt | Pink | | Pink |
| Urobilinogen | 2.9% p-diethylaminobenzaldehyde | Pink | Deeper Pink | Deepest |
| | | | | Pink |
| Specific | 2.8% Bromthymol blue; | Blue | Blue Green | Khaki- |
| Gravity | 1.2% polyacid | | | Yellow |

For example, a first reagent may be provided in the liquid-absorbent layer (e.g. the absorbent body 24 indicated in FIG. 1) or lateral flow device facilitating the detection of a single substance whose presence or concentration is abnormal. Desirably, the test panel 320 includes indicia 330 to indicate where to look for a test result. As shown by way of example in FIG. 3, the indicia may take the form of alphanumeric characters. Of course, color coding or a numbering system may be used. It is possible that a number of reagents may be provided in the liquid-absorbent layer in the form of a sequence (not shown). The results of the interaction between each reagent may be portrayed on the test panel 320. For example, as seen in FIG. 3, the result of bilirubin detection is shown to indicate if bilirubin is at an acceptable level. In addition, the result of hydration detection may be positioned on the test panel to indicate the urine concentration, and thus, if hydration is at an acceptable level. Further, a fullness gauge 332 may be present to show “capacity,” meaning how much absorbent capacity the garment has remaining, or how much urine the garment has absorbed. For example, see U.S. Pat. No. 4,834,733 issued to Huntoon et al. on May 30, 1989, incorporated by reference.

Such, physical measurements (e.g. fullness gauge) such as by capillary action will likely not interfere with chemical reagents or with other physical measurements. Of course, when more than one chemical reagent is used, it is either separated from other chemical reagents such as by a lateral flow device, or not capable of interfering with other chemical reagents.

It is further contemplated that a wearer or caregiver may need a portable color chart (not shown in the figures) for rapid comparison with the colors present on test panel 320 or on garments removed from persons. It should be noted, however, that a color chart will often be unnecessary in certain embodiments of the present invention.

Providing these reagents in the liquid-absorbent layer or on lateral flow devices in the manner described above allows caregivers to screen for such conditions as urinary tract infections, glycosuria from diabetes mellitus or other causes of hyperglycemia, hematuria, and kidney or biliary abnormalities, among others. This screening may be accomplished quickly and easily during the course of normal care.

### Array

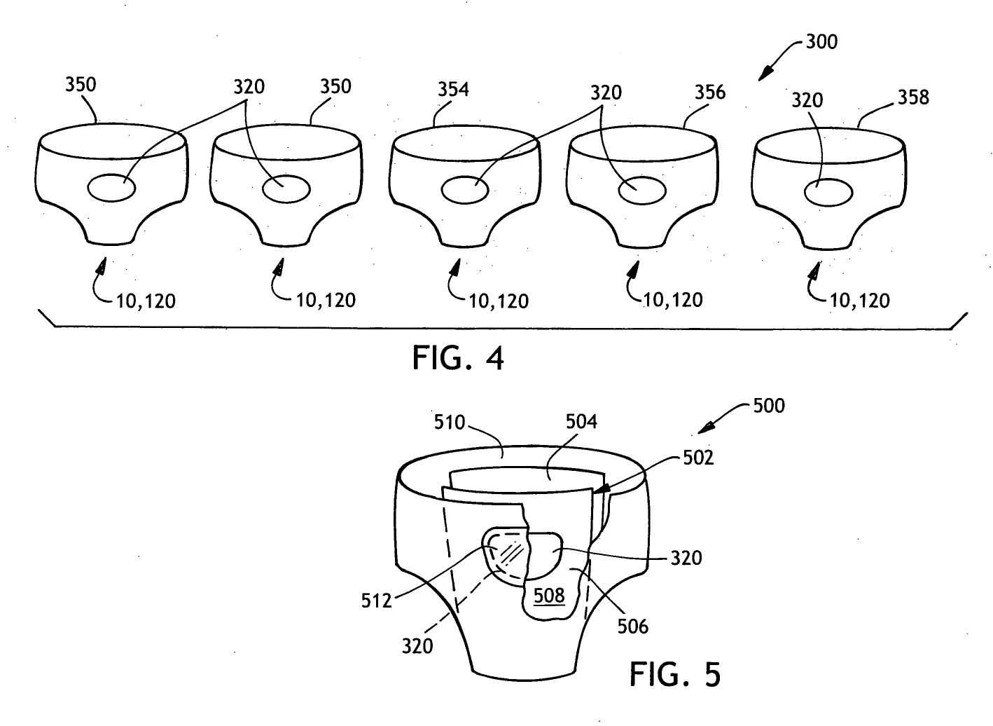

Shown in FIG. 4 is one embodiment of an array of disposable absorbent articles or garments, referenced as garments 350, 352, 354, 356, and 358, are configured to fit the lower region of a human torso to collect urine and feces. The garments 350-358 in array 300 have a liquid permeable bodyside liner, an outer cover, and an absorbent body positioned between the bodyside liner and the outer cover as described above for the garments portrayed in FIGS. 1 and 2. While the array 300 is shown as having a diaper 10 or a pant 120, the array may comprise other embodiments of the garments. For example, the array might be entirely composed of diapers 10, entirely composed of pants 120, or a combination of pants and diapers.

The garments of the array 300 may be all the same size or different sizes, or may be designed to fit persons in the same or different life stages as defined herein. In the alternative, each of the garments in this embodiment of the array 300 may differ only by what reagents are included in each of the garments in the array. Each of the garments may detect one or more of the following substances or physical conditions as described herein: bilirubin, urobilinogen, leukocytes, nitrites, red blood cells, glucose, protein, ketones, hydrogen ions, ion concentrations, phenylalanine, and the remaining liquid-capacity of the garment. However, it is further contemplated that other urine substances relating to metabolic or physiological conditions may be detected.

In one embodiment, the array 300 includes only two garments, a first garment 350 having a first reagent, configured to detect in urine, an abnormal level of a substance associated with a first medical condition. The medical condition may be a urinary tract infection, a biliary abnormality, dehydration, hematuria, glycosuria, proteinuria, or ketonuria. There is a second garment 352 having a second test panel responding to a second reagent configured to detect, in urine, an abnormal level of a substance associated with a second medical condition. The medical condition may be a urinary tract infection, a biliary abnormality, dehydration, hematuria, glycosuria, proteinuria, ketonuria, or any other disease or condition that can be detected by a reagent capable of being disposed in the liquid absorbing layer of a garment and reacting with urine to provide a result to a wearer or caregiver.

In another embodiment, the first garment 350 may be smaller in size than the garment 352. For example, the first garment 350 may be sized to fit a newborn as this category of persons can be susceptible to jaundice, and may detect bilirubin (or urobilinogen), or in the alternative, hydration level. The second garment 352 may be sized to fit an infant, toddler, child, or adult as defined herein. The second garment 352 may detect glucose or ketones. Both garment 350 and 352 may show whether or not an insult of urine has occurred, and may show urine capacity.

To differentiate between sizes, terminology such as “small,” “medium,” “large,” “extra large,” and “extra-extra large” may be used; however, these terms are used in a relative sense only and should not be interpreted to be fit a particular life stage (e.g. weight range) unless specifically limited by the claims. However, it is noted that each size will fit into the definition of a life stage. For example, a “small” garment my fit a toddler, whereas a “medium” garment may fit an adult. In another example, a small garment may fit a newborn, a medium garment may fit an infant, and a large garment may fit a toddler.

In another embodiment, the array 300 may include not only the first and second garments 350 and 352, but also include a third garment 354 with a test panel 320 having a third reagent configured to detect, in urine, an abnormal level of a substance associated with a medical condition after the reagent contacts the urine. The medical condition may be a urinary tract infection, a biliary abnormality, dehydration, hematuria, glycosuria, proteinuria, or ketonuria. The third reagent may be different from the first and second reagents used in this embodiment of array 300.

The third garment 354 may be larger in size than the first or second garments 350, 352, or may be the same size as either of those garments. In one embodiment, the first garment 350 is smaller than the second garment 352, and the second garment 352 is smaller than the third garment 354.

In yet another embodiment, the array 300 may include not only the first, second, and third garments 350-354, but also include a fourth garment 356 with a test panel 320 having a fourth reagent configured to detect, in urine, an abnormal level of a substance associated with a medical condition after the reagent contacts the urine. The medical condition may be a urinary tract infection, a biliary abnormality, dehydration, hematuria, glycosuria, proteinuria, or ketonuria. The fourth reagent may be different from the first, second, and/or third reagents used in this embodiment of array 300.

The fourth garment 354 may be larger in size than the first, second, or third garments 350-354, or may be the same size as any of those garments. In one embodiment, the first garment 350 is smaller than the second garment 352, the second garment 352 is smaller than the third garment 354, and the third garment 354 is smaller than the fourth garment 356.

In a further embodiment, the array includes a fifth garment 358 to make an array of five garments. The fifth garment 356 includes a test panel 320 having a fifth reagent configured to detect, in urine, an abnormal level of a substance associated with a medical condition after the reagent contacts the urine. The medical condition may be a urinary tract infection, a biliary abnormality, dehydration, hematuria, glycosuria, proteinuria, or ketonuria. The fifth reagent may be different from the first, second, third and/or fourth reagents used in this embodiment of array 300.

The fifth garment 358 may be larger in size than the first, second, third or fourth garments 350-356, or may be the same size as any of those garments. In one embodiment, the first garment 350 is smaller than the second garment 352, the second garment 352 is smaller than the third garment 354, and the third garment 354 is smaller than the fourth garment 356, and the fourth garment 356 is smaller than the fifth garment 358.

Each of the garments 350-358 may include more than one chemical reagent for the detection of other medical abnormalities or conditions. For instance, one of the garments may detect both glucose and blood, either of which may indicate that the wearer has a medical condition.

In addition, each of the garments 350-358 may be able to provide a physical measurement in addition to a chemical reagent. For example, one or more of the garments in an array 300 may include a device such a fullness gauge 332 that will indicate the amount of liquid that has been absorbed by the diaper, or the amount of capacity remaining in the diaper to absorb more liquid, or both.

Desirably, in each of the garments in an array 300, the result of the reaction between the reagent and urine is portrayed by a visible marker or indicia 330 that is displayed on a test panel 320. Though not necessary, it is desirable to have the test panel 320 located on the front panel of an absorbent article, as shown in FIG. 4 where the test panel 320 is shown on a front panel of each garment 350-358. Specifically, the front waist region 122 (FIG. 2) or front panel 19 (FIG. 1) has a test panel 320 on the outer or garment-facing surface of the garment. However, it is contemplated that the test panel 320 may be located on the rear or crotch portion of a garment, and may possibly be on the bodyfacing surface or liner of the garment. Test panel 320 may have any shape or aesthetic features. Further, the test panel 320 may be composed of multiple small panels (not shown) and not a single body as shown. Indicia 330 may be included on the test panel to relay information to the user so that the results of the interaction between the chemical reagent and substance may be determined by the garment wearer or a caregiver. When the results are merely shown on the test panel, it may be said that the test panel has a reagent located in the test panel 320, even if the reagent is located in the absorbent member of a product. Indicia 330 may also provide information not due to a chemical reaction, but due to a physical reaction such as wicking or capillary action (e.g. a fullness gauge 332 to indicate how much liquid or urine the garment has absorbed).

In another embodiment of the present invention there is an array 300 of disposable absorbent garments. Each garment in the array 300 generally has a liquid permeable bodyside liner, an outer cover, and an absorbent body positioned between the bodyside liner and the outer cover as described previously with respect to the garments described in relation to FIGS. 1 and 2. A small garment 302 is adapted to fit an infant weighing about 0.45 kilograms to about 5.44 kilograms, and having a test panel 320 including a first reagent. The small garment 302 may have a diaper configuration as shown, or may be a pant or pad configuration. The reagent may be adapted to react to a biliary abnormality, and be selected from one or more of the following chemicals: dichloroaniline diazonium salt and diethylaminobenzaldehyde. A second reagent may be included that will detect wetness.

Desirably, in addition to the small garment, there may be a medium garment 303 adapted to fit a human weighing more than about 5 kilograms. Optionally, the medium garment may be adapted to fit a human weighing about 5 kilograms to about 20 kilograms. Further, the medium garment may be adapted to fit a human weighing about 15 kilograms to about 30 kilograms. The medium garment has a test panel 320 including a reagent. The reagent may be adapted to react to detect hydration, and optionally, a second reagent may be adapted to detect yet another physical condition.

Array 300 may further include a large garment adapted to fit a human weighing about 20 to about 40 kilograms. The large garment includes a test panel 320 including a reagent. The reagent may be adapted to react to detect hydration, and optionally, a second reagent may be adapted to detect yet another physical condition.

Optionally, array 300 may further include an extra large garment adapted to fit a human weighing about 50 to about 200 kilograms. The extra large garment includes a test panel 320 having a reagent adapted to react to a substance relating to hydration.

In yet another embodiment, the extra large garment may be most suitable for patients that are inclined to suffer from diabetes. The reagent may be adapted to detect glycosuria, and may be selected from one or more of the following chemicals: glucose oxidase, peroxidase, and potassium iodide. Optionally, a second reagent may be adapted to detect a urinary tract infection, and may be selected from one or more of the following chemicals: naphthyl ester, diazonium salt, arsenilic acid, and tetrahydrobenzo (h) quinolin.

Referring now to FIG. 6, in yet another embodiment of the present invention, there is an array 500 of articles used for detecting a substance in urine. The array includes a pad 502 having a liquid permeable bodyside liner 504, an outer cover 506, and an absorbent body (not shown) positioned between the bodyside liner 504 and the outer cover 506 as is known in the art. For example, see, U.S. Pat. No. 5,651,778 issued Jul. 29, 1997 to Melius et al., incorporated by reference to the extent it is consistent with the present invention. The pad 502 is used to collect urine, and includes a test panel 320 located desirably on the outer cover 506, at a garment facing surface 508. The test panel 320 has at least one chemical reagent associated therewith capable of detecting the substance in urine as described herein. Further included in the array 500 is a garment 510 used to support the pad 502 against the body of a wearer.

The garment 510 may have an underwear configuration and be disposable (as defined herein) or washable and reusable. Thus, the garment 510 may be made from fabrics used to make durable underpants (e.g. cotton, nylon, silk, and the like) or may be made from a nonwoven material (e.g. the spunbond-meltblown-spunbond, spunbond, and the like). The garment 510 includes a window 512 on the front of the garment through which the test panel 320 may be observed. Window 512 is made from a clear, flexible material such as vinyl. In operation, the test panel 320 is aligned with window 512 so that any test panel results may be observed through window 512.

While we have shown and described certain preferred embodiments, it is understood that the invention may be embodied otherwise than as herein specifically illustrated and described. Other embodiments that are apparent to those of ordinary skill in the art are also within the scope of the invention. For instance, the invention may extend to a disposable pad that could be affixed into a garment. The garment may include a window through which a pad test panel could be observed. The garment may be disposable or washable and reusable. Accordingly, the scope of the present invention is not intended to be limited by the foregoing, but rather by reference to the appended claims.

What is claimed is:

1. An array of disposable absorbent garments for wearing about the lower torso; the array comprising:
a small garment adapted to fit a human of a small size and comprising a first test panel, the first test panel comprising a first small reagent;
a medium garment adapted to fit a human of a medium size that is greater than the small size, and comprising a second test panel, the second test panel comprising a first medium reagent;
wherein the first small reagent is adapted to react to a first substance, and the first medium reagent is adapted to react to a second substance, and wherein the first substance is significantly different than the second substance.

2. The array of claim 1 further comprising a large garment adapted to fit a human weighing about 20 to about 40 kilograms, comprising a third test panel, the third test panel comprising a first large reagent.

3. The array of claim 2 wherein the first large reagent is adapted to detect a urinary tract infection.

4. The array of claim 2 wherein the first large reagent is adapted to detect dehydration.

5. The array of claim 2 further comprising a second large reagent located in the second test panel.

6. The array of claim 5 wherein the second large reagent is adapted to detect glycosuria.

7. The array of claim 6 wherein the second large reagent is selected from the group consisting of glucose oxidase, peroxidase, and potassium iodide.

8. The array of claim 1 wherein the first small reagent is adapted to detect a biliary abnormality.

9. The array of claim 1 wherein the first small reagent is adapted to detect dehydration.

10. The array of claim 1 wherein the small garment further comprises a second small reagent located in the second test panel.

11. The array of claim 1 wherein the first medium reagent is adapted to detect glucose.

12. The array of claim 1 wherein the first medium reagent is adapted to detect dehydration.

13. The array of claim 1 wherein the medium garment is adapted to fit a human weighing about 5 kilograms to about 25 kilograms.

14. The array of claim 1 wherein the small garment is adapted to fit a human weighing about 0.4 to about 5.5 kilograms.

15. An array of disposable absorbent garments for absorbing urine, the array comprising:
a first garment comprising a first reagent configured to detect, in urine, an abnormal level of a first substance associated with a biliary abnormality after said first reagent contacts said urine; and
a second garment comprising a second reagent configured to detect, in urine, an abnormal level of a substance associated with a second medical condition after said second reagent contacts said urine, said medical condition being selected from the group consisting of: (a) urinary tract infection, (b) hematuria, (c) glycosuria, (d) proteinuria, (e) dehydration, and (f) ketonuria.

16. The array of claim 15, further comprising a test panel located on the first garment and the second garment, the test panel adapted to display a result of any interaction between particular substances and the first reagent and the second reagent.

17. The array of claim 16 wherein the test panel is further adapted to display if an insult has occurred.

18. The array of claim 16 wherein the test panel is located on a garment-facing surface of a front panel of the first garment and the second garment.

19. The array of claim 15 wherein the first garment is further configured to detect and display on a test panel, urine volume that has been absorbed by the first garment.

20. The array of claim 15 wherein the second garment is further configured to detect and display on a second test panel, urine volume that has been absorbed by the second garment.

21. The array of claim 20 further comprising a third garment with a third test panel comprising a third reagent configured to detect, in urine, an abnormal level of a substance associated with a medical condition after said reagent contacts said urine, said medical condition being selected from the group consisting of: (a) urinary tract infection, (b) hematuria, (c) glycosuria, (d) proteinuria, (e) dehydration, and (f) ketonuria; and wherein the third reagent is different from the first and second reagent.

22. The array of claim 15 wherein the first substance or second substance is selected from the group consisting of bilirubin and urobilinogen.

23. The array of claim 15 wherein the first substance or second substance is selected from the consisting of leukocytes and nitrites.

24. The array of claim 15 wherein said first substance or second substance comprises red blood cells.

25. The array of claim 15 wherein said first substance or second substance comprises glucose.

26. The array of claim 15 wherein the first substance or second substance comprises a protein.

27. The array of claim 15 wherein the first substance or second substance comprises ketones.

28. The array of claim 15 wherein the first substance or second substance comprises hydrogen ions.

29. The array of claim 15 wherein the first substance or second substance comprises phenylalinine.

30. The array of claim 15 wherein the test panel is further adapted to display the relative volume of a urine insult with respect to absorption capacity of the corresponding disposable first or second garment.

31. The array of claim 15 further comprising a test panel located on a front region of the first garment and the second garment.

32. An array of articles used for detecting a substance in urine comprising:
a pad having a liquid permeable bodyside liner, an outer cover, and an absorbent body positioned between the bodyside liner and the outer cover, the pad adapted to collect urine and comprising a test panel, the test panel comprising a chemical reagent capable of detecting the substance; and
a garment adapted to support the pad against the body, the garment including a window through which the pad test panel may be observed.
